(19) United States
(12) Patent Application Publication
(10) Pub. No.: US 2004/0154044 A1
(43) Pub. Date: Aug. 5, 2004

(54) Transgenic cell and animal modeling ige-mediated human allergic responses and use thereof

(76) Inventors: Alexandre Fraichard, Lyon (FR); Yadine Cherifi, Lyon (FR); Kader Thiam, Lyon (FR)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/472,050

(22) PCT Filed: Mar. 15, 2002
(86) PCT No.: PCT/FR02/00933

(30) Foreign Application Priority Data
Mar. 15, 2001 (FR) 01/03520

(51) Int. Cl. A01K 67/027; C12N 5/06

(52) U.S. Cl. 800/6; 800/14; 435/326

(57) ABSTRACT

The invention relates to a transgenic non-human animal cell characterized in that it expresses at least one nucleotide sequence coding for at least one of the chains of human receptors of the fragment F_{c} of IgE immunoglobulins (F_{c}εR)) and a nucleotide sequence coding fo a human origin, characterized in that the murine gene coding for the chain F_{c}ε)R) of the human receptor is inactive. The invention also relates to a corresponding transgenic animal and a method for understanding physiopathological elements involved in immediate hypersensitivity and/or inflammatory mechanisms. The invention further relates to a method for screening active compounds on interactions between human IgE's and the receptors thereof.

The present invention relates to the field of biology and more particularly to the field of transgenic animals. The invention relates to a non-human transgenic animal cell, characterized in that it expresses at least one nucleotide sequence coding for at least one of the human receptor chains of F_{c} fragment of the IgE (F_{c}εR) and a nucleotide sequence coding for the heavy chain of IgE, of which at least all or part of the F_{c} is of human origin and, characterized in that the murine gene coding for the F_{c}εR chain homologous to said F_{c}εR chain of the human receptor is inactive. The invention relates to the corresponding transgenic animal as well as to the method for demonstrating an allergen. The invention also relates to a screening method for a compound for understanding the pathophysiological elements implicated in the immediate hypersensitivity mechanisms.

Allergies and clinical manifestations that are associated with them comprise a growing problem in public health, especially in the Western countries. The allergic reaction is a hypersensitivity reaction occurring immediately after contact with an antigen (allergen) upon a second exposure to this antigen. This hypersensitivity reaction is only the consequence of an inadequate expression of immune responses of the organism culminating in inflammatory reactions and tissue damage.

Coombs and Gell have defined four type of hypersensitivity (I, II, III and IV); type I, or immediate, hypersensitivity corresponds to the allergic reaction. The clinical manifestations of type I hypersensitivity, still called atopy, comprise for example asthma, rhinitis, eczema, hay fever, urticaria. Type I hypersensitivity is linked to a response of the IgE against the antigens without toxicity per se such as pollen, for example.

A cascade of events develops between the first mucus membrane contact with the allergen and the appearance of allergic symptoms connected with the second contact with the same allergen. First of all, a localized release of IgE is seen at the site of entry of the allergen in the organism, such as the mucus membranes and/or the regional lymph nodes. The production of the IgE by the B cells involves the participation of cells presenting the antigen (PCAg), T “helper” cells and the stimulation of IgE producing B cells. The IgE produced locally sensitize the surrounding mastocytes; the interaction between the IgE immunoglobulins and the mastocytes and the basophils via their F_{c}εR cellular receptors constitutes the first event in the allergic response. The mastocytes thus activated expands the mediators such as histamine, heparin, leukotrienes, for example, which directly produce the allergic symptoms (Ishizaka, 1989). The IgEs produced in excess pass into the circulation, where they sensitize the circulating basophils then the tissue mastocytes of the entire organism. The serum levels of IgE are often elevated in allergic disorders and considerably increased in the parasitoses. Apart from the mastocytes and the basophils, a certain number of other cells carrying receptors for the F_{c} extremity of the IgE (F_{c}εR) intervening in the immediate hypersensitivity mechanisms in the human beings. Thus, the number of circulating monocytes have F_{c}εR is also more elevated in certain atopics, in particular in those having severe atopic eczema; these monocytes, when they are armed with the IgE become potentially cytotoxic. In like manner, the alveolar macrophages can be sensitized by IgEs and, in the presence of allergens, release enzymes. These phenomena are capable of playing an important role in allergic respiratory diseases. The eosinophils and platelets also carry F_{c}εR. When they are sensitized by IgEs, the cells are found to considerably increase their cytotoxic properties vis-a-vis certain parasites, including the schistosomes. The Langerhans cells also express IgE receptors. In contrast, in the animal model, in particular in the mouse, only two cell types appear to be involved in the immediate hypersensitivity mechanisms; these are mastocytes and basophils.

These different cells sensitized by immune complexes comprise IgEs, assuring important functions in allergic disorders, insofar that they reinforce all kinds of pharmacologically active mediators capable of stimulating or controlling allergic reactions. When the IgEs are fixed on the F_{c}εR of the mastocytes, basophils, eosinophils, degranulation can be triggered by the cross linking of IgEs, involving that of the F_{c}εR. The immunologic agents triggering activation by the F_{c}εR disrupt the mastocyte membrane, which provokes the entry into the cells of calcium ions that are essential to degranulation. Entry of calcium ions has essentially two consequences: firstly, the release of preformed mediators, the main one of which is histamine and among which there is also heparin, proteolytic enzymes such as tryptase and β-glucosamimidase and chemotactic factors and activators like CPA, NCF and PAF and, secondly, the induction of synthesis of mediators newly formed from arachidonic acid leading to the production of prostaglandin and leucotriene. These mediators act directly on the surrounding tissues and at the level of the lungs, provoking an immediate bronchoconstriction, edema of the mucous membranes and hypersecretion that results in the asthmatic crisis.

There are different types of IgE receptors in the mastocytes: (i) a tetrameric receptor comprised of two α chains and two β chains having high affinity to IgE (F_{c}εR) that binds the monomeric IgE immunoglobulin (Kinet, 1992; Metzger, 1992) and (ii) two receptors also having a weak affinity to IgG (F_{c}γRII and F_{c}γRIII) that bind both of the IgG and IgE immune complexes (Takizawa et al., 1992). The central role of the F_{c}εRI in the allergic reaction ahs been shown by Dombrowic et al. (1993).

The allergic response in mammals comprises a complex phenomenon that results in the action of one or a plurality of allergens, of a plurality of genes coding for structural and/or functional proteins. Although recent advances have been made in the understanding of the metabolic cascades and pathways leading to allergic manifestations. The allergic phenomena remain relatively poorly understood, which makes difficult the development of preventive and/or curative treatments such as, the development of inhibitors of the IgE receptors, for example.

There is thus an urgent need to find efficacious inhibitors of the allergic response. To date, the discovery of inhibitors has been made difficult by the lack of in vitro and in vivo screening models for such compounds. Although the in vivo model has the advantage of showing systemic alterations at the level of the entire animal, the utilization of animals such as mice, for example, for studying the human allergic response continues to be of limited interest because such a model only imperfectly reproduces the mechanism of the type I immediate hypersensitivity reaction in the human being; in fact, in the mouse the IgE receptors are expressed solely in the mastocytes and the basophils, whereas in the human being their expression is detected in mastocytes, basophils, eosinophils, monocytes, Langerhans cells. In order to extenuate the drawbacks of transgenic mice expressing the human F_{c}εRI receptor have been created. Accordingly, Dombrowic et al. (1993) have shown that a mouse knock-out (KO) for the α chain of the F_{c}εRI receptor is unable to develop passive anaphylaxis but that the anaphylactic response can be reconstructed in the mouse obtained using embryonic stem cells (ES). knock-out (KO) for the α chain of the murine F_{c}εRI and express the alfa chain of human F_{c}εRI. The model developed by Dombrowic et al. nevertheless has a certain number of limitations because (i) the integration of the DNA sequence of the α chain of human F_{c}εRI is done randomly which can affect the expression of other genes, (ii) the transgene is present in multi-copy form (approximately 300) which can also affect the rate of expression of the receptor and regulation of its expression and finally (iii) the high affinity of the F_{c}εRI receptor to IgE cannot respond to the same stimuli as its murine homologue. Likewise, the WO 95 15376 application suggests using a mouse whose human F_{c}εRI receptor is humanized in order to screen the candidate molecules that inhibit the allergic response. Although this patent application suggests replacing by genetic screening in the embryonic stem cells (ES cells), the murine F_{c}εRI gene with its human equivalent, this application does not have any experimental data tending to demonstrate that the inventors succeeded in obtaining such transgenic mice. Likewise, the system suggested in the WO 95 13376 application has the drawback of not having reproduced in their entirety the parameters of receptor—IgE interaction. In fact, in the system, immunoglobulin E is of murine origin; such an immunoglobulin does not have the same affinity for its receptor as its human equivalent. Therefore, such a model, if it in fact exists, would only imperfectly reproduce the human situation.

In order to extenuate the drawbacks of the study models and screening of inhibitors of the allergic reaction of the prior art and in order to accelerate discovery of efficacious inhibitors of allergic reactions, the inventors propose providing a transgenic non-human animal cell, characterized in that it expresses at least one nucleotide sequence coding for at least one of the human receptor chains on the F_{c} fragment of the immunoglobulins and a nucleotide sequence coding for the heavy chain of an immunoglobulin, of which all or part of the F_{c} fragment is of human origin and, characterized in that the animal gene coding for the chain homologous to the said chain of the human receptor is inactive. According to one preferred embodiment of the invention, the transgenic non-human animal cell expresses at least one nucleotide sequence coding for at least one of the human receptor chains on the F_{c} fragment of the IgE (F_{c}εR) immunoglobulins and a nucleotide sequence coding for the heavy chain of an IgE immunoglobulin, of which at least all or part of the F_{c} fragment is of human origin and is characterized in that the animal gene coding for the F_{c}εR chain homologous to the said F_{c}εR chain of the human receptor is inactive.

Inactivation of said gene or inactive gene is understood to mean a gene whose expression is null or strongly inhibited in the said cell. This absence of expression or this strong inhibition is translated either by an absence of or a negligible quantity of corresponding transcribed RNA in the cell or by the presence of a truncated transcript or by an absence of or negligible quantity of the corresponding protein, or by a corresponding truncated and/or inactivated protein; in other words, deprived of biological activity.

F_{c}εR receptor is understood to mean any cellular receptors participating in the immediate hypersensitivity mechanism and capable of linking the immunoglobulin Es by their F_{c} fragment. Among the F_{c}εR receptors, F_{c}εRI (Kinet, 1992; Metzger, 1992), F_{c}γRII, F_{c}γRIII (Takizawa et al., 1992), F_{c}εRII/CD23 (Conrad, 1990), Mac 2 (CPB35, εPB) Cherayil et al., 1989; Fnigeri et al., 1992; Truong et al., 1993).

Receptor chain is understood to mean the sub-unit or one of the sub-units of the receptor of the F_{c} fragment of the IgE when said receptor is monomeric or multimeric, respectively. Preferably, the chain of the human receptor according to the invention that is expressed, is the chain that codes for the binding site of the receptor of the F_{c} fragment. Preferably, the F_{c} fragment receptor of the E immunoglobulins is the F_{c}εRI. The F_{c}εRI is a tetrameric complex of an α chain, a β chain and two y chains linked by a disulfide bond (Knet, 1992; Metzger, 1992). Only the completely assembled tetrameric complex is expressed at the cell surface (Blank et al., 1989). According to the present invention, said F_{c}εRI receptor chain is chosen from the a chain, the β chain, and the γ chain. Preferably, it is an α chain, because it contains entirely the binding site of the receptor to the F_{c} fragment. In fact, a mouse whose gene coding for the α chain of the F_{c}εRI was inactivated homozygotically does not express the F_{c}εRI at the level of the cell surface (WO 95 15376. Alternatively, said F_{c}εRI receptor chain is the β chain that is replaced by the human β chain that is replaced by the human β chain (Kuster et al., 1992). According to another embodiment, said F_{c}εRI receptor chain is the γ chain that is replaced by the human γ chain. Nevertheless, according to another embodiment of the invention, it can advantageously express at least one, at least two, at least three or the four human chains of the F_{c}εRI complex instead of their murine homologues.

The E immunoglobulin or IgE is an immunoglobulin, whose serum concentration is very low. The ε chain as an elevated molecular point (72.5 kDa) and comprises approximately 550 amino acids distributed in four constant domains (C_{ε}1, C_{ε}2, C_{ε}3, C_{ε}4). Enzymatic cleavage of the IgE by papain releases a 5S fragment having a molecular weight of approximately 98 kDa corresponding to the F_{c} fragment. This F_{c} fragment comprises the majority of the specific determinants of the IgE molecule. The IgE molecule according to the invention comprises at least all or part of a human F_{c} fragment. Preferably, the transgene according to the invention corresponds to the C_{ε}3, C_{ε}4 domains of the F_{c} fragment of the IgE. Optionally, the entirety of the F_{c} fragment of the IgE is of human origin or the IgE according to the invention is human.

The invention can be realized in any mammalian cell capable of homologous recombination. Preferably, it is a rodent cell, especially of the mouse, the rat, the hamster, and the guinea pig. Preferably, they are mouse cells. Alternatively, it is a primate cell, with the exception of the human being, such as the simians, the chimpanzee, macaque, and baboon. It can also be a bovine, caprine, ovine, porcine, especially of the dwarf pig, equine, especially of the horse, lagomorph, especially the rabbit, cell.

The cells according to the invention correspond to any animal cells, preferably mammalian cells, with the exception of human cells. Examples of competent mammalian cells for recombination thus comprise fibroblasts, endothelial cells, epithelial cells as well as cells usually cultured in the laboratory such as Hela, the CHO cells (Chinese hamster cells), for example.

The cells according to the invention can be defined functionally as being capable of realizing homologous recombination of the exogenous DNA fragment(s) having sequence homologies with an endogenous cellular DNA sequence. Such cells naturally contain the endogenous recombinases or have been genetically modified by containing it or for containing the compounds necessary for realizing the recombination of the DNA.

Preferably, of the cells according to the invention, all types of cells naturally expressing the receptor binding the F_{c} portion of the IgE (F_{c}εR) and/or expressing the IgE immunoglobulins can be mentioned such as, for example, the cells of the immune system or certain neuronal cells. Of the immune system cells the T lymphocytes, NK cells, K cells, B-lymphocytes, mastocytes, macrophages, monocytes, neutrophils, eosinophils, basophils, platelets, monocytes of dendritic cells, Langerhans cells can be mentioned non-exhaustively. Certain of these cells express the F_{c}εR receptors having a particularly elevated affinity (k_{A}=10¹⁰ M⁻¹): these are mastocytes and basophils. Other cells such as monocytes, macrophages, eosinophils, as well as the platelets expressing the F_{c}εR fragment, express the F_{c}εR fragment with a much weaker affinity (k_{A}=10¹⁰ M⁻²). It is also worth mentioning the cells that, under certain culture conditions or after differentiation or genetic manipulation are capable of expressing the receptor binding the F_{c} portion of the IgE (F_{c}εR) and/or expressing the IgE immunoglobulins. One can mention the hematopoietic stem cells, the neuronal stem cells, the omnipotent or pluripotent embryonic stem cells (ES cells). These stem cells can differentiate into a cell expressing the transgenes according to the invention such as, for example, the cells of the immune system or the neuronal cells. Stem cells are understood to mean all types of multipotential or pluripotential undifferentiated cells that can be cultured in vitro over extended periods of time without loosing their characteristics and that are susceptible to differentiated into one or several cell types, when they are placed under defined conditions of culture. Accordingly, when the cell according to the invention is an ES cell or a hematopoietic cell, one can envisage inducing its differentiation into different types of cells capable of expressing the transgenes such as, for example, the T lymphocytes, the NK cells, the K cells, the B lymphocytes, the mastocytes, the macrophages, the monocytes of the dendritic cells, the Langerhans cells. When it is necessary to use embryonic stem cells (ES) for the production of the transgenic animal according to the invention, for example, a cell line of ES cells can be used or the embryonic cells can be obtained freely using a host animal according to the invention, in general a mouse, a rat, a hamster, a guinea pig. Such cells are cultivated on a layer of appropriate feeder fibroblasts or on gelatin in the presence of appropriate growth factors such as leukemia inhibiting factor (LIF for leukemia inhibiting factor)

In the context of the present invention, a transgenic is defined as a cell comprising a transgene. A transgene is defined as a sequence of exogenous nucleic acids or an exogenous gene or a nucleotide sequence of the genetic material that has been or is going to be inserted artificially into the genome of a mammal, in particular into a mammal cell cultured in vitro or into a living mammal cell or that is going to be used in said episomal cell. Preferably, the nucleotide sequence according to the present invention comprises at least one sequence capable of being transcribed or transcribed and transduced into protein. The transgene can be cloned into a cloning vector that makes it possible to assure its propagation in a host cell and/or optionally into an expression vector for assuring the expression of the transgene. Recombinant DNA technologies utilized for construction of the cloning vector and/or expression according to the invention are known cells and commonly used by the specialist in the filed. The standard techniques are utilized for cloning, DNA isolation, amplification and purification; the enzymatic reactions involving DNA ligase, DNA polymerase, restriction endonucleases are practiced according to the manufacturer's recommendations. These techniques and the others are generally practiced according to Sambrok et al. (1989). The vectors include plasmids, retroviruses and other animal viruses, artificial chromosomes, such as YAC, BAC, HAC and other analogous vectors.

The methods for generating transgenic cells according to the invention are well known to the specialist in the field (Gordon et al., 1989). Various techniques for transfection mammalian cells have been described (for a review, see Keon et al., 1990). The transgene according to the invention optionally comprises in a vector, whether linearized or not, or in the form of a fragment of the vector, can be introduced into the host cell using standard methods such as, for example, micro-injection into the nucleus (U.S. Pat. No. 4,873,191), transfection by precipitation of calcium phosphate, lipofection, electroporation (LO, 1983), heat shock, transfection using cationic polymers (PEG, polybrene, DEAE dextran, etc.), viral infection (Van der Putten et al., 1985), sperm (Lavitrano et al., 1989).

According to a preferred embodiment of the invention, the transgenic cell according to the invention is obtained by genetic targeting of the transgene(s) at the level of one or several sequences of the genome of the host cell. More precisely, the transgene is inserted stably by homologous recombination at the level of homologous sequences in the genome of the host cell. When it is a question of obtaining a transgenic cell with a view of producing a transgenic animal, the host cell is preferably an embryonic stem cell (ES cell) (Thompson et al., 1989).

Genetic targeting represents the directed modification of a chromosomal locus by homologous recombination using an exogenous DNA sequence with the targeted endogenous sequence. Different types of genetic targeting are distinguished. Accordingly, genetic targeting can be utilized for modifying the expression of one or a plurality of endogenous gene(s) or for replacing an endogenous gene with an exogenous gene or for placing an exogenous gene under the control of regulatory elements of genetic expression of a particular endogenous gene that remains active. In this case, the genetic targeting is called knock-in (KI). Alternatively, the genetic targeting can be utilized for reducing or eliminating expression of one or a plurality of genes. Thus, this is genetic targeting called knock-out (KO) (see Bolkey et al., 1989).

In order to produce the homologous recombination it is necessary that the transgene containing at least one DNA sequence comprise at least one portion of the targeted gene, with any desired genetic modifications and likewise DNA regions of homology having the target locus, preferably numbering two, situated at both ends of the portion of the target gene. Homologous regions of DNA or homologous DNA sequences are understood to mean two DNA sequences that, after optimal alignment and after comparison, are identical for approximately 75% of the nucleotides, at least approximately 80% of nucleotides, usually at least approximately 90% to 95% of nucleotides and, preferably, at least approximately 98% to 99.5% of the nucleotides. Percent identity between two nucleic acid sequences in the context of the invention is understood to mean a percentage of identical nucleotides between two sequences to be compared, obtained after the best alignment, this percentage being purely statistical and the difference between the two sequences being randomly distributed and over its entire length. Best alignment or optimal alignment is understood to mean the alignment for which the percentage of identity determined as hereinbefore defined is the highest. The comparisons of the sequences between two sequences of nucleic acids are conventionally practiced by comparing these sequences after having optimally aligned them, said comparison being realized by segment or by comparison window in order to identify and compare the local regions of sequence similarity. Optimal alignment of the sequences for comparison can be done, in addition to manually, by means of the local homology algorithm described by Smith and Waterman (1981), by means of the local homology algorithm described by Neddleman and Wunsch (1970), by the methods of similarity research described by Pearson and Lipman (1988), by means of software programs utilizing these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimal alignment, the BLAST program is preferably used with the BLOSUM 62 matrix. The PAM or PAM250 matrices can also be used. The percentage of identity between two nucleic acid sequences is determined by optimally comparing the two aligned sequences, the sequence of nucleic acids or amino acids to be compared can comprise additions or deletions relative to the reference sequence for an optimal alignment between these two sequences. The percentage of identity is calculated by determining the number of identical positions for which the nucleotide or the amino acid residue is identical between the two sequences by dividing this number of identical positions by the total number of positions compared and by multiplying the result obtained by 100 for obtaining the percentage of identity between these two sequences. Nucleic acid sequences having a percentage of identity of at least 85%, preferably at least 90%, 95%, 98% and 99% after optimal alignment with a reference sequence, the reference nucleic sequences is understood to mean, relative to the reference nucleic sequences, certain modifications as, in particular, a deletion, a truncation, an elongation, a chimeric fusion, and/or a substitution, especially pointwise, and in which the nucleic acid sequence has at least 85%, preferably at least 90%, 95%, 98% and 99% of identity after optimal alignment with the reference nucleic sequence.

The length of the regions of homology is partially dependent on the degree of homology. This is due to the fact that a reduction in the quantity of homology results in a reduction of the frequency of homologous recombination. If there are regions of non-homology between the portions of homologous sequences, it is preferably that this non-homology is not spread over the entire portion of homologous sequence but rather in discrete portions thereof. At all events, the lower the degree of homology the longer the region of homology must be in order to facilitate the homologous recombination. Although as few as 100% homology of 14 bp can be sufficient for realizing homologous recombination in bacteria, the longest portions of homologous sequences are preferred, in general. These portions are at least 250 bp, 500 bp, 750 bp, 1,000 bp, 1,500 bp, 1,750 bp, preferably at least 2,000 bp for each portion of homologous sequence. According to the invention, the DNA fragments are of any size. The minimum size required is subordinate to the necessity of having at least one region of homology sufficiently long to facilitate homologous recombination. The DNA fragments have a size of at least approximately 2 kg, preferably at least around 3 kb, 5 kb, 6 kb.

The transgene is not limited to a particular DNA sequence. The DNA sequence can be of a purely synthetic origin (for example, routinely produced using a DNA synthesizer) or can be derived from mRNA sequences by reverse transcription or can be derived directly from genomic DN. When the DNA sequence derives from RNA sequences by reverse transcription, it may or may not contain all or part of the non-coding sequences such as the introns, depending on whether or not the corresponding RNA molecule has undergone splicing wholly or in part. Preferably the DNA utilized for carrying out the homologous recombination comprises genomic DNA rather than cDNA. In fact, important cis-regulatory sequences present in the introns, distal regions, and promoter regions can be present. The sequences deriving from genomic DNA coding generally at least for a portion of the gene but capable alternatively of coding for non-transcribed regions or regions of a non-rearranged genetic locus such as the immunoglobulin loci or the T-cell receptor loci. Generally, the genomic DNA sequences include a sequence coding for a RNA transcript. Preferably, the RNA transcript codes for a polypeptide. Preferably, it is a question of all or part of the F_{c} fragment of the immunoglobulins, preferably IgE, or a human receptor chain on the F_{c} fragment of the immunoglobulins, preferably F_{c}εR.

The transgene according to the invention can contain appropriate regulatory sequences for directing and controlling the expression of said polypeptides in the appropriate cell type(s). Preferably, the transgenes are deprived of the necessary regulatory sequences for directing and controlling expression in one or several appropriate cell type(s); the transgenes are positioned after homologous recombination under the control of the endogenous animal sequences regulating the expression of the animal gene that is preferably inactivated by the homologous recombination event and integration of the human gene. Alternatively, the expression of one of the transgenes can be placed under the control of human exogenous regulatory sequences and the other under the control of endogenous murine regulatory sequences.

The transgene can also be as small as several hundreds of cDNA base pairs or as large as a hundred thousand base pairs of a genic locus comprising the exonic—intronic coding sequence and the regulatory sequences necessary for obtaining a temporally—spatially controlled expression. Preferably, the recombinant DNA sequence of a size between 1.5 kb and 1,000 kb. Whatever it is, the recombinant DNA segments can be less than 2.5 kb and greater than 1,000 kb.

The transgene or the DNA sequence of the present invention is preferably in native form; that is, derived directly from an exogenous DNA sequence naturally present in an animal cell. This native DNA sequence can be modified, for example, by insertion of restriction sites necessary for cloning and/or by insertion of site-specific recombination sites (lox and flp sequences). In the alternative, the DNA sequence of the present invention can have been created artificially in vitro using the recombinant DNA techniques, by combining, for example, portions of genomic DNA and cDNA. These are chimeric DNA sequences.

The DNA sequence according to the invention, native or chimeric, can be mutated by utilizing the techniques well known to the specialist in the field. Thus, the nucleotide sequence coding for at least one of the human receptor chains on the F_{c} fragment of immunoglobulins, preferably IgE, and the nucleotide sequence coding for all or part of the heavy chain of the immunoglobulins, preferably IgE, capable of being changed either in their coding or non-coding sequence. The gene introduced can thus be a wild type gene having a natural polymorphism or a genetically manipulated sequence, for example having deletions, substitutions or insertions in the coding or non-coding regions. For the coding sequences, these mutations can affect the amino acid sequences. Accordingly, the DNA sequence coding for all or part of the human F_{c} fragment of the IgE utilized for producing the homologous recombination can be obtained by directed mutagenesis of the corresponding murine F_{c} fragment.

When the gene introduced is a coding sequence, it is generally operationally linked to elements controlling genic expression. One nucleic sequence is “operationally linked” when it is placed in functional relation with another nucleic acid sequence. For example, a promoter or an activator (enhancer) is operationally linked to a coding sequence, if it affects the transcription of said coding sequence. Concerning the regulatory sequences of the transcription, “operationally linked” means that the linked DNA sequences are contiguous and when it is a question of linking two regions coding for proteins, contiguous and in reading phase. For the “switch” sequences of the immunoglobulins “operationally linked” means that the sequences are capable of effecting the recombination “switch.”

When the cells have been transformed by the transgene, they can be transformed by the transgene, it can be cultured in vitro or can even be utilized for producing transgenic animals. After transformation, the cells are seeded on a feeder layer and/or in an appropriate medium. The cells containing the construction can be detected by utilizing a selective medium. After a time sufficient for allowing the colonies to propagate, they are collected and analyzed for determining if an event of homologous recombination and/or an integration of the construction is produced. In order to practice the screening of clones having undergone homologous recombination positive and negative markers, also known as selection genes, can be inserted into the homologous recombination vector. Different selection systems of cells having accomplished the homologous recombination event have been described; the first system described can be mentioned, which utilizes positive/negative selection vectors (Mansour et al., 1988; Capecchi, 1989).

Selection gene is understood to mean a gene that makes it possible for cells having it to be selected specifically for or against the presence of a corresponding selection agent. To illustrate this concept, a gene coding for antibiotic resistance can be utilized as a positive selection marker that makes it possible for a host cell to be selected positively in'the presence of the corresponding antibiotic. A variety of positive and negative markers are known to the specialist in the field (for a review, see U.S. Pat. No. 5,627,059). This selection gene can be situated either within or outside of the linearized transgene. When the selection gene is positioned within the transgene; in other words, between the 5′ and 3′ ends of the transgene, it can be present in the form of a genic entity distinct from the reporter gene according to the invention. In this case, the selection gene is operationally linked with the DNA sequences allowing control of its expression; in the alternative, the selection gene can be placed under the control of the sequences regulating expression of the said reporter gene. These sequences, well known to the specialist in the field, correspond especially to the promoter sequences, optionally to the enhancer sequences and to transcription termination signals. Optionally, the selection gene can constitute a fusion gene with the reporter gene. Said fusion gene is thus linked operationally with the DNA sequences making possible control of the expression of said fusion gene. According to another embodiment of the invention, the selection gene is situated at the 5′ and 3′ ends of the transgene such that if a homologous recombination event is produced, the selection gene is not integrated into the cellular genomic DNA; in this case, the selection gene is a negative selection gene (for a review, see the U.S. Pat. No. 5,627,059).

Said positive selection gene according to the invention is chosen preferably from among the antibiotic resistance genes. Of the antibiotics neomycin, tetracycline, ampicillin, kanamycin, tetracycline, ampicillin, kanamycin, chloramphenicol, carbenicillin, genticin, puromycin can be mentioned non-exhaustively. The resistance genes corresponding to these antibiotics are well known to the specialist in the field; by way of example, the neomycin gene renders the cells resistant to the presence of the G418 antibiotic in the culture medium. The positive selection gene can also be selected from the HisD gene, the corresponding selective agent being histidiol. The positive selection gene can also be selected from the gene for guanine phophoribosyl transferase (GpT), the corresponding selective agent being xanthine. The positive selection gene can also be selected from the gene for hypoxanthine phophoribosyl transferase (HPRT), the corresponding selective agent being hypoxanthine.

Said negative selection gene according to the invention is preferably chosen from the gene for 6-thioxanthine or thymidine kinase (TK) (Mzoz et al., 1993), the genes coding for the bacterial or viral toxins such as, for example, the Pseudomonas exotoxin, diphtheria toxin (DTA), cholera toxin, bacillary anthrax toxin, Pertussus toxin, Shigella Shiga toxin, the toxins of Escherichia coli, colicine A, d-endotoxin. cytochrome p450 of the rat and cyclophosphamide (Wei et al., 1994), the purine nucleoside phosphorylase of Escherichia coli (E. coli) and the 6-methylpurine deoxyribonucleoside (Sorcher et al., 1994), the cytosine deaminases (Cdase) or uracil phophoribosyl transferase (UPRTase) can also be mentioned, which can be utilized with 5-fluorcytosine (5-F_{c}).

The selection marker(s) utilized to make it possible to identify the homologous recombination events can consequently affect genic expression and can be eliminated, if necessary, by using site-specific recombinases such as Cre recombinase specific to the lox sites (Sauer, 1994; Rjewsky et al., 1996; Sauer, 1998) or FLP specific to the FRT sites (Kilby et al., 1993).

The positive colonies; that is, those continuing the cells in which at least one homologous recombination event is produced, are identified by Southern blotting analysis and/or by PCR techniques. The rate of expression, in the isolated cells or the cells of the transgenic animal according to the invention, of the mRNA corresponding the transgene can also be determined by the techniques including Northern blotting, in situ hybridization, RT-PCR. Likewise, the animal cells or tissues expressing the transgene can be identified using an antibody directed against the reporter protein.

The positive cells can then be utilized for carrying out manipulations on the embryo and especially the injection of cells modified by homologous recombination in the blastocysts. As concerns the mouse, the blastocysts are obtained from 4 to 6 week superovulated females. The cells are trypsinated and the modified cells are injected into the blastocell of a blastocyst. After injection, the blastocysts are introduced into the uterine horn of the pseudo-gestating females. The females are allowed to reach term and the resulting litters are analyzed in order to determine the presence of mutant cells having the construction. The genotypic or phenotypic analysis differing between the cells of the newborn fetus and the cells of the blastocyst or the ES cells make it possible to detect the chimeric neonates. The chimeric fetuses are then reared to adulthood. The chimeras or chimeric animals are animals in which only one sub-population or cells has the altered genome. The chimeric animals having the modified gene or genes are generally bred with each other or with a wild-type animal in order to obtain heterozygous or homozygous offspring. The heterozygous males and females are then bred in order to generate homozygous animals. Unless otherwise indicated, the transgenic animal according to the invention includes stabile changes in the nucleotide sequence of the cells of the germ line.

According to another embodiment of the invention, the non-human transgenic cell according to the invention can be used as a nucleus donor cell in the framework of a nucleus transfer or nuclear transfer. Nuclear transfer is understood to mean the transfer of the nucleus of a living vertebrate donor cell, of an adult or fetal stage organism, into the cytoplasm of an enucleated recipient cell of the same species or of a different species. The transferred nucleus is reprogrammed for directing the development of the cloned embryos that can then be transferred into carrier females for producing the fetuses and neonates, or utilized for producing cells of the internal cellular mass in culture. Different nuclear cloning techniques are capable of being used; among these, those forming the object of patent applications WO 95 17500, WO 97 07668, WO 97 07669, WO 98 30683, WO 99 01163, WO 99 37143 can be mentioned non-exhaustively.

According to one preferred embodiment of the invention, the nucleotide sequence coding for at least on of the human receptor chains on the F_{c} fragment, preferably F_{c}εR, and/or all or part of the human F_{c} fragment of the heavy chain of the immunoglobulins, preferably the IgE immunoglobulins, is stably integrated into the genome of said cell. This integration into the genome of said cell of said human gene(s) coding for one of the chains of the human F_{c} fragment receptors of the immunoglobulins, preferably the F_{c}εR and/or the integration of all or part of the human F_{c} fragment of the heavy chain of the immunoglobulin, especially the IgE immunoglobulins, is realized by homologous recombination and comprises a knock-in; it is practiced at the level of the said homologous animal gene(s) at the said human gene(s) coding, respectively, for one of the F_{c} fragment receptors of the immunoglobulins, preferably the F_{c}εR receptor, and for all or part of the nucleotide sequence coding for the F_{c} fragment of the heavy chain of the immunoglobulins, preferably the IgE immunoglobulins, said integration provoking the inactivation of said corresponding homologous animal gene.

The nucleotide sequence that codes for at least one of the human F_{c} fragment receptor genes of the immunoglobulins, preferably F_{c}εR, is operationally linked to sequences regulating expression, sad sequences controlling the expression of said sequence in the cell; preferably, it these are endogenous animal sequences regulating the transcription of the homologous gene coding for the human F_{c} fragment receptor chains of the immunoglobulins. In the alternative, these are endogenous human sequences regulating the expression of the homologous gene coding for the human F_{c} fragment receptor of the immunoglobulins.

According to a preferred embodiment, the nucleotide sequence that codes for the heavy chain of an immunoglobulin, preferably an IgE, is the endogenous animal gene, with the exception of the sequence coding for all or part of the F_{c} fragment of said immunoglobulin that is of human origin, said sequence coding for all or part of the F_{c} fragment having been integrated into said gene by homologous recombination (knock-in). In the alternative, the nucleotide sequence coding for the heavy chain of an immunoglobulin is the human gene coding for the heavy chain of an immunoglobulin, said human gene being integrated by homologous recombination (knock-in) into the genome of said cell, at the level of said homologous animal gene, said integration provoking the inactivation of said homologous animal gene.

The nucleotide sequence that codes for the heavy chain of an IgE of which at least all or part of the F_{c} fragment is of human origin and is operationally linked to the endogenous animal sequences for regulating the expression of said gene of the human IgE heavy chain, said sequences controlling the expression of said nucleotide sequence in said cell. In the alternative, the sequences for regulating expression are the endogenous human sequences for regulation of transcription of said gene of the heavy chain of human immunoglobulins, preferably the human IgEs.

According or one embodiment, the exogenous gene according to the invention is deprived of the elements for regulating of genic expression and is placed under the control of the endogenous elements for regulating expression of the target gene. Accordingly, according to a preferred embodiment of the invention, the gene of the α chain of the human gene of the F_{c}εRI is placed in a murine cell under the control of the elements regulating expression of the gene of the α chain of the murine F_{c}εRI and in the same murine cell the gene of the heavy chain of the IgE comprising all or part of the F_{c} fragment of human origin is placed under the control of the elements for regulation of expression of the gene of the murine IgE heavy chains.

Elements for regulation of genic expression is understood to mean all of the DNA sequences implicated in the regulation of genic expression; e.g., essentially the regulatory sequences of transcription, splicing, translation. Of the DNA transcription regulatory sequences the minimal promoter sequences, the upstream sequences (for example, the SP1, IRE for interferon regulatory element, etc.), the activator sequences (enhancers), any inhibitor sequences (silencers), insulator sequences (insulators), splicing sequences can be mentioned.

The elements controlling genic expression allow either a constitutive, ubiquitous, inducible, specific expression of a cell type (tissue specific) or specific to one developmental stage. These elements may or may not be heterologous to the organism, or may or may not be naturally present in the genome of the organism. It is evident that as a function of the desired result, the specialist in the field will choose and adapt the elements of regulation of expression of the genes. Preferably, the regulatory sequences of expression are those of the exogenous gene. Accordingly, according to a preferred embodiment of the invention, where the exogenous coding sequence is the sequence coding human F_{c}εRI, the promoter and the other regulatory sequences deriving from the human promoter and the human regulatory sequences of the F_{c}εRI gene.

In the cell according to the invention, the nucleotide sequence coding for the heavy chain of an immunoglobulin is preferably the endogenous animal gene, with the exception of the sequence coding for all or part of the F_{c} fragment of said immunoglobulin that is of human origin, said sequence coding for all or part of the F_{c} having been integrated into said gene by homologous recombination (knock-in). Preferably, it is an IgE immunoglobulin. In the alternative, the nucleotide sequence coding for the heavy chain of an immunoglobulin is the human gene coding for the heavy chain of an immunoglobulin, said human gene being integrated by homologous recombination (knock-in) into the genome of said cell, at the level of said homologous animal gene, said integration provoking the inactivation of the said homologous animal gene. Preferably it is an IgE immunoglobulin

The non-human transgenic cell and/or the transgenic animal according to the invention is obtained by introducing, simultaneously or staggered in time, at least one transgene coding for a human receptor chain of the immunoglobins, preferably F_{c}εR, and a transgene coding at least for the F_{c} fragment of the heavy chain of human immunoglobulins, preferably IgE, into a zygote or an early embryo of a non-human animal. Optionally, it may be interesting to introduce a transgene coding for all or part of the light chain of the human immunoglobulins, preferably IgE. Introduction of these different transgenes into the cell according to the invention may be practiced simultaneously or staggered in time.

According to a preferred embodiment, the double transgenic cell according to the invention can be obtained directly by simultaneous introduction of the DNA fragments necessary to the homologous recombination into said cell by utilizing methods favoring the co-transformation of multiple DNA molecules. The cells are thus selected for the double expected recombination event by utilizing an adapted system of selection. In the alternative, the double transgenic cell according to the invention can be obtained by practicing the homologous recombination events separately and staggered in time. Accordingly, the cell, after introduction of a first homologous recombination vector, is selected for the first homologous recombination event by utilizing an adapted selection system; this newly transgenic cell is then transformed using a second homologous recombination vector, then selected for the second homologous recombination event by utilization of an identical or different selection system. Optionally, this double transgenic cell can then be transformed using a third homologous recombination vector, then selected for the third homologous recombination event by utilizing an identical or different selection system, and so on. In the alternative, the doubly, triply, or multiply transgenic cell according to the invention can be obtained by successive crossing of singly transgenic animals. For example, the doubly transgenic cell can be obtained by crossing of two homozygous singly transgenic animals; it can be obtained by crossing, then selection of two heterozygous singly transgenic animals or by crossing and selection of a homozygous singly transgenic with a heterozygous singly transgenic animal.

Preferably, said human gene coding for one of the F_{c} fragment receptor chains of the immunoglobulins, preferably F_{c}εR, and all or part of the human F_{c} fragment of the heavy chain of the immunoglobulins, preferably IgE, are stably integrated into the genome of said cell. Stabile integration is understood to mean the insertion of the transgene into the genomic DNA of the cell according to the invention. The transgene so inserted is then transmitted to the cellular offspring.

Said cell and said animal can be obtained by utilizing different strategies. Preferably, the strategy consists of practicing the knock-in of all or part of the sequence coding for the F_{c} constant fragment of the heavy chain of the IgE. The knock-in can relate, for example, to the entirety of the constant fragment or only to the C_{ε}3, C_{ε}4 portion of the F_{c} fragment of the IgE interacting with the F_{c}εR receptor. According to a preferred embodiment, only the F_{c} fragment of the murine gene coding for the heavy chain of the IgEs is replaced by gene targeting (knock-in) by the F_{c} fragment of the human gene of the heavy chain of the IgE. According to this preferred embodiment, the immunoglobulin or the entire group of immunoglobulins, preferably the IgEs, produced by the transgenic cell or, respectively, by the transgenic animal according to the invention will have all or part of the humanized F_{c} fragment. Such an animal is capable of rearranging the segments of the genes of the humanized immunoglobulins, preferably IgE, for producing a primary antibody response and capable of developing a secondary antibody response by somatic mutations of the rearranged immunoglobulin genes, preferably IgE. According to this preferred embodiment, the repertoire of immunoglobulin heavy chains, preferably of the IgEs of the transgenic animal according to the invention corresponds to the natural repertoire of the animal. Such an animal will be capable of reacting to murine allergens.

According to a second embodiment, it may be interesting that the cell or animal according to the invention expresses a repertoire of human immunoglobulin heavy chains, preferably heavy chains of human IgE or optionally a repertoire of human immunoglobulins, preferably human IgE. In order to do this, it is necessary to introduce the repertoire of heavy chains of human immunoglobulins or human IgE instead of that of the mouse. Different technologies can thus be used (see, for example, EP 546 073, WO 95 15376). Preferably, the animal expresses a repertoire of human immunoglobulins and is thus capable of reacting to human antigens. Accordingly, the cell according to the invention comprises a nucleotide sequence coding for the heavy chain of the immunoglobulins and a nucleotide sequence coding for the light chain of the immunoglobulins, preferably IgE. These nucleotide sequences are comprised of non-rearranged human genomic DNA. In the case of the heavy chain, the transgene contains the entirety or a part of the members of all of the six known V_{H} (or several hundreds of possible V_{H} segments), the D segments (dozen segments), the J segments (four segments), as well as the constant ε region (Berman et al., 1998). The transgenic cell line or the transgenic mouse expressing one such transgene correctly expressed the heavy chain of human immunoglobulins, preferably IgE, as well as a large repertoire of variable regions for triggering an immune response to the majority of antigens. In the case of the light chain, the transgene contains the entirety or a part of the members of all of the known V_{H} families, the J segments, as well as the constant ε region. In the alternative, the transgene coding for the heavy chain and, optionally, the light chain of the immunoglobulin according to the invention, preferably IgE, can be generated by intracellular recombination in vivo according to the technique described in EP 546 073.

Finally, the animal according to the invention can constitutionally or inducibly express a unique humanized immunoglobulin according to the invention, preferably an IgE, coded by a unique rearranged gene. This immunoglobulin, preferably an IgE, being directed against a particular allergen, the transgenic animal constitutes a study and screening model for inhibitor receptors on the F_{c} fragment of the immunoglobulin directed against this specific allergen.

Finally, the human or humanized gene coding for an IgE immunoglobulin can be a mini-gene. A mini-gene is understood to mean a DNA sequence, generally of a size smaller than 150 kb, generally comprises between 25 and 100 kb and containing at least one variable V segment, a J segment, a constant C, region and when it is a mini-gene of the heavy chain of a D segment.

According to another embodiment of the invention, the cell is characterized in that said human gene coding for an immunoglobulin, preferably an IgE, is present in episomal form in said cell and in that said homologous animal gene is inactive in said cell. According to this embodiment, said homologous animal gene is inactivated by homologous recombination (knock-out).

It can also be of interest to instantaneously detect if the transgenic cell or transgenic animal according to the invention develops an allergic response following exposure to one or a plurality of allergens. This is reason for which the cell according to the invention is characterized in that its genome contains in addition at least one reporter gene operationally linked to one or a plurality of sequences regulating inducible expression following simulation of the F_{c}εR receptor and or stimulation of the synthesis of IgE. The sequence(s) regulating the expression is or are chosen from the promoter of the CD23 gene or the interleukin 4 (I1-4) gene and any other gene, whose expression is induced following a stimulation of the F_{c}εR receptor and/or IgE synthesis. According to a preferred embodiment, the transgenic cell can be obtained using a transgenic animal obtained by crossing a transgenic animal, whose genome contains a reporter gene operationally linked to one or a plurality of sequences of regulation of expression inducible following stimulation of the F_{c}εR and/or stimulation of the primary synthesis of IgE, and a transgenic animal having at least one human F_{c} fragment receptor chain of the immunoglobulins, preferably IgE, and a nucleotide sequence coding for all or part of the human F_{c} fragment of IgE. This transgenic animal is also one of the objects of the present invention. In the alternative, the reporter gene can be present in episomal form in an expression vector in said cell.

It can also be of interest to simultaneously detect the type of polarization of the immune response and/or the type of effector function induced by one or a plurality of allergens. This is the reason for which the present invention proposes introducing also into the genome of the present cell according to the invention at least one transgene coding at least for one reporter protein, whose expression is correlated with the expression of at least one protein naturally produced by said cell and specifically of one type of polarization of the immune response, especially Th1 and Th2, and/or an effector function of the immune response. According to this particular embodiment of the invention, the cell according to the invention comprises in addition a first transgene coding for a first reporter protein, said first transgene being integrated by homologous recombination (knock-in) at the level of an endogenous gene coding for a specific protein of a first type of polarization of the immune response, such as Th1, without invalidating the expression of said endogenous gene, the expression of said first transgene being correlated with the expression of said endogenous gene; and (ii) a second transgene coding for a second reporter protein, different from said first reporter protein, said second transgene being integrated by homologous recombination (knock-in) at the level of an endogenous gene coding for a specific protein of a second type of polarization of the immune response, such as Th2, without invalidating the expression of said endogenous gene, the expression of said second transgene being correlated with the expression of said endogenous animal gene. Preferably, this non-human transgenic cell is characterized in that the type Th1 specific protein of polarization of the immune response is γ-IFN, the specific protein of the type Th2 polarization of the immune response is interleukin-4 (IL-4), the said first transgene codes for the GFP reporter protein and the second transgene codes for the RFP reporter protein. Such a multi-transgenic cell is preferably obtained from cells derived from a multi-transgenic animal obtained by successive breeding of transgenic animals.

A reporter gene is understood to mean a gene that allows cells having this gene to be detected specifically, following expression of same; in other words, to be distinguished from other cells that do not carry this marker gene. Said reporter gene according to the invention codes for a reporter protein chosen from the group comprised of auto-fluorescent proteins, such as green fluorescence protein (GFP), enhanced green fluorescence protein (EFGP), yellow fluorescent protein (YFP), blue fluorescence protein (BFP), red fluorescence protein (RFP), as well as the variants of these fluorescence proteins obtained by mutagenesis in order to generate a fluorescence of a different color. Said reporter gene codes also for any enzyme detectable by fluorescence, phosphorescence or visibly by a histochemical method on living cells or any other method of cellular analysis, or by microscopy. The following can be mentioned non-exhaustively: β-galactosidase (β-GAL), β-glucoronidase (β-GUS), alkaline phosphatase, especially placental alkaline phosphatase (PLAP), alcohol dehydrogenase, especially Drosophila alcohol dehydrogenase (ADH), luciferase, especially firefly luciferase, choramphenicol acetyl transferase (CAT), growth hormone (GH).

The present invention relates also to the transgenic animal comprising at least one cell according to the invention. Transgenic animal is understood to mean a non-human animal, preferably a mammal, chosen from the group comprising rodents, and especially the mouse, rat, hamster, guinea pig. The mouse is particularly preferred because its immune system has been studied in depth and especially the genetic organization of loci of light and heavy chains of the immunoglobulins. However, the rat constitutes an excellent alternative for modeling immediate and inflammatory hypersensitivity reactions, because the physiologic response is very much more pertinent it the rat than in the mouse. Alternatively, the transgenic animal is chosen from among breeding animals and especially the porcines, preferably the dwarf-pig, ovines, caprines, bovines, equines, especially the horse, and the lagomorphs, especially the rabbit. The transgenic animal can also be chosen from among the primates, especially the simians, baboons, macaques, chimpanzees, with the exception of the human being.

The transgenic animal according to the invention comprises at least one cell, whose genome comprises at least one exogenous nucleic acid sequence present either as an extrachromosomal element or stably integrated into the chromosomal DNA. Preferably, the set of these cells and especially the germ cell line are transgenic.

Considering the genetic polymorphisms present in the population, it may be of interest to analyze a physiological or behavioral characteristic that the transgenic animals according to the invention and especially the transgenic mice according to the invention may have different genetic bases. Accordingly, the mice according to the invention can be selected in consanguine murine lines (inbred) 129v, 12901a, C57B16, BalB/C, DBA/2 but also in the non-consanguine lines (outbred) or hybrid lines.

Preferably, the animal according to the invention is characterized in that it expresses an immunoglobulin repertoire, preferably IgE, functional after an exposure to at least one allergen, said IgEs having at least all or part of the F_{c} fragment of human origin and/or at least one of the human F_{c} fragment receptor chains of the immunoglobulins, preferably IgE.

It is also one of the objects of the present invention to provide an in vitro method for demonstrating an allergen and/or determination of the allergizing power of said allergen, characterized in that it comprises the steps of (i) placing said allergen in contact with a cell according to the invention; (ii) determination if an immediate cellular hypersensitivity and/or inflammatory reaction is produced, and (iii) optionally, qualitative and/or quantitative evaluation of the allergic reaction. Likewise, the present invention seeks to provide an in vivo method for demonstrating an allergen and/or determination of the allergizing power of said allergen, characterized in that it comprises the steps of (i) placing said allergen in contact with said animal according to the invention; (ii) determination if a immediate hypersensitivity and/or inflammatory reaction is produced, and (iii) optionally qualitative and/or quantitative evaluation of the allergic reaction.

For the purposes of the present invention, an allergen is understood to mean those compounds capable of inducing an immediate hypersensitivity and/or inflammatory reaction. Of the allergens, one can mention non-exhaustively the allergens derived from pollen, fungi (Aspergillus, Candida, Alternaria, etc.), bacterial (food bacteria, lactobacteria, etc.), mites (Dermatophagoides pteronyssinus, Dermatophagoides farinae, etc.), the allergens derived from debris of animal skin, feces and fur (for example, Fel dI feline alergy), allergens of insect origin, food allergens (eggs, milk, meat, seafood, beans, cereals, fruits, legumes, chocolate, yogurt, etc.) the household allergens (mites etc.), the parasite allergens (nematodes, schistosomes, helminthes, etc.), the mitogens, pathogenic agents, or one of their constituents, of viral, bacterial, parasitic, fungal, mycoplasmic, drugs (for example, penicillin and insulin), excipients, vaccines and vaccinal components, chemical compounds (such as the isocyanates, ethylene oxide, latex, etc. for example).

Placing a specific allergen in contact with a cell or an animal according to the invention can be done in diverse ways such as, for example, classical infection by a pathogenic micro-organism, or via a biological delivery vector (mosquito, tick, bacterium, virus and parasites or common recombinant agent, naked DNA, etc.), by inhalation, in an aerosol, through food intake. Experimentally, the allergen can be brought into contact with the animal by systemic administration, in particular by intravenous, intramuscular, intradermal route, cutaneous contact, by oral route or, if it is a cell culture, in the culture medium.

The present invention also provides a method for screening a compound that modulates, preferably inhibits, the immediate hypersensitivity and/or inflammatory reaction in human beings. This method is characterized in that it comprises the steps of (a) placing a cell and/or an animal according to the invention in contact with an allergen responsible for triggering the immediate hypersensitivity and/or inflammatory reaction and, simultaneously or staggered in time, with said compound; (b) placing a cell and/or an animal according to the invention in contact with a said allergen of step a); (c) determination and qualitative, optionally quantitative evaluation if an immediate hypersensitivity and/or inflammatory reaction is produced and then comparison of said immediate hypersensitivity and/or inflammatory reactions triggered in a) and b); then (d) identification of the compound that selectively modulates the immediate hypersensitivity and/or inflammatory reaction.

In the methods according to the invention, said determination and/or evaluation of said immediate hypersensitivity and/or inflammatory reaction is realized by measuring the IgE level synthesized by the cell according to the invention and or by the serum IgE level of the animal according to the invention. Alternatively, and preferably, said determination and/or evaluation of the allergic reactions is done by the detection and/or measurement of the rate of expression of said reporter gene.

The present invention also relates to the utilization of a composition comprising a compound modulating the immediate hypersensitivity and/or inflammatory reaction and a pharmaceutically acceptable vehicle as a medicine for the preventive and/or curative treatment of a human being or of an animal requiring such treatment, characterized in that the ability of said compound to selectively inhibit or activate the immediate hypersensitivity and/or inflammatory reaction is determined by (a) placing a cell and/or an animal according to the invention in contact with an allergen responsible for triggering the immediate hypersensitivity and/or inflammatory reaction and simultaneously or staggered in time with said compound; (b) placing a cell and/or an animal according to the invention in contact with said allergen of step a); (c) determination and qualitative, optionally quantitative evaluation if an immediate hypersensitivity and/or inflammatory reaction is produced and then comparison of said immediate hypersensitivity and/or inflammatory reactions triggered in a) and b); then (d) identification of the compound that selectively modulates the immediate hypersensitivity and/or inflammatory reaction. Modulatoin of the immediate hypersensitivity and/or inflammatory reaction is understood to mean an inhibition, a diminution but also an activation. The compounds modulating the immediate hypersensitivity and/or inflammatory reaction obtained by the screening process and the composition according to the invention are utilized as a medicine for the treatment of pathologies chosen from the group comprising asthma, eczema, hay fever, urticaria, the allergies, atopic dermatitis, chronic inflammatory diseases of the intestine (CIDI) and/or of the colo-rectum such as, for example, Crohn's disease, parasitic diseases in which the IgE response is known to be protective, especially the helminthic parasitic diseases (infections with Schistosoma mansoni, and Nippostrongylus filaria). Acceptable pharmaceutical vehicle is understood to mean any type of vehicle usually employed in the preparation of pharmaceutical and vaccinal compositions; that is, a diluent, synthetic or biological vector, a suspension agent such as an isotonic or buffered saline solution. Preferably, these compounds are administered systemically, in particular by intravenous, intramuscular, intradermal or by oral route. Their optimal routes of administration, doses and galenic forms can be determined according to the criteria generally taken into consideration in establishing a treatment adapted to a patient as, for example, age or body weight of the patient, the severity of his general condition, tolerance to the treatment and the confirmed secondary effects, etc. When the agent is, for example, a polypeptide, an antibody, an antagonist, a ligand, a polynucleotide, for example, an antisense composition, a vector, for example an antisense vector, it can be introduced into the host tissues or cells in a certain number of ways, including viral infection, micro-injection or vesicular fusion. Jet injection can also be used for intramuscular injection.

The immediate hypersensitivity and/or inflammatory reaction is chosen from among systemic anaphylaxis, cutaneous anaphylaxis, asthma, eczema, rhinitis, atopic dermatitis, chronic inflammatory diseases of the intestines (CIDI) and/or colo-rectum, such as Crohn's disease, for example, the parasitic diseases in which the IgE response is known to be protective, especially the helminthic parasitic diseases (infections with Schistosoma mansoni, and Nippostrongylus filaria), and the food allergies and household dust allergies.

Another object of the invention is the utilization of a cell or an animal according to the invention for analyzing and/or studying the molecular, biological, biochemical, physiological and/or pathophysiological mechanisms of the immediate hypersensitivity and/or inflammatory reaction. By utilizing the transgenic cells or animals according to the invention, it is possible to identify ligands or substrates that modulate the interactions between the human immunoglobulin E and its F_{c}εR receptor, preferably F_{c}εRI, implicated in the type 1 hypersensitivity phenomena. A large number of tests can be done for this purpose that include behavior tests, determination of the localization of drugs following their administration. As a function of the type of test that is to be developed, either an entire animal, or cells derived from said animal. These cells can be either isolated freely from the animal or can be immortalized in culture or by multiplying the passages, either by transforming the cells by using viruses such as the SV40 virus or the Epstein-Barr virus.

Other characteristics and advantages of the invention will become apparent when reading the description together with the examples represented hereinafter. In these examples, reference will be made to the following examples.

### EXAMPLES

Materials and Methods

1.1 Humanization of the α Chain of the F_{c}εRI Murine Gene

The region coding the 5 exons of the murine gene or a part of the coding region (exon IV, for example) is replaced, by homologous recombination, by its human equivalent, hF_{c}εRI, described by Dombrowicz et al. (Dombrowicz et al., 1993). The arms of the homologous recombination are cloned using the murine sequence available in the databanks (Genbank NM 01084). These fragments are combined with the gene coding for the α chain of the human receptor and with the negative and positive selection genes (HPRT), this latter being flanked by loxP sites. The unique enzymatic sites are introduced in order to make possible the linearization of the homologous recombination vector as well as screening and the selection of the recombinant clones by the PCR technique and/or Southern blot.

1.2 Humanization of the Murine Gene Coding for the Constant Region of IgE

The expression of the recombinant proteins in the baculovirus (Vangelista et al., 1999) has demonstrated that the Cε3 constant region of the human IgE would correspond to the minimum structure necessary to the interaction with its high affinity receptor, F_{c}εRI. As a result and in the same fashion as in the above paragraph, the genomic sequence coding for a part or the totality of the constant region of the murine IgE is replaced by homologous recombination, with a part (Cε3) or the totality of the constant region of human IgE.

1.3 Culture of ES Cells and Electroporation

The ENS and E14TG2a cell lines (Hooper et al., 1987) are cultured as described by Koller and Smithies (Koller and Smithies, 1989). The homologous recombination vectors, prepared in the form of plasmid DNA, are amplified, purified and controlled according to the classically described methods (Sambrook J. et al., 1989). Electroporation of the ES cells is practiced in culture media containing JnM of linearized homologous recombination under the conditions hereinbefore described. The transfected colonies are selected in virtue of the presence of the positive/negative resistance marker, HPRT. The homologous recombination event is enriched by the presence of the negative selection marker (gene coding for thymidine kinase or for diphtheria toxin, sub-unit A) The selected clones are the object of a deletion of the HPRT gent by transfection of a vector for expression of the Cre recombinase (Gu H et al., 1994). The recombinant clones are selected in the presence of 6-TG.

1.4 Analysis by PCR and Southern Blot

The homologous recombination event is detected by PCR and/or Southern blot. For PCR, the segments are localized on the outside of the short homologous arm and in the resistance gene. The signal due to the amplification can be detected only in the case of homologous recombination. In the other cases, no signal is detectable.

The structure of the positive clones after PCR is verified by Southern blot. The DNA extracted from recombinant clones are subjected to one or a plurality of enzymatic digestions and the nucleic transfers are hybridized using two probes; on being external to the homologous recombination vector and the other being specific to the positive selection marker. In the same way, the clones having undergone excision of the HPRT gene are the object of screening by PCR and by Southern blot. In this case, the primers chosen are situated at both ends of the selection marker. The clones having undergone the action of the recombinase give a smaller signal. In the case of the Southern blot, the probe used corresponds to a portion of the HPRT gene. The positive clones are characterized by the absence of a signal.

1.5 Production of Transgenic Mice

The selected clones are injected into blastocytes of C57BL/6 mouse blastocytes in order to generate chimeric mice (Koller and Smithies, 1989). The presence of the transgenes in the offspring is verified by Southern blot using the tails of the mice (Miller et al., 1988). For each model, the transgenic homozygotes are produced by crossing heterozygotes.

The two models of transgenic mice can be produced using the same clone of the ES clones transfected by two homologous recombination vectors or even separately and in parallel.

### REFERENCES

Berman et al. (1998) EMBO J. 7: 727-738

Blank et al. (1989) Mol. Immunol. 26: 107-114

Bolkey et al. (1989) Ann. Rev. Genet. 23: 199-225

Capecchi (1989) Science, 244: 1288-1292

Cherayil et al. (1989) J. Exp. Med. 170: 1959-1972

Conrad (1990) Ann. Rev. Immunol. 8: 623-645

Dombrowicz et al. (1993) Cell 75: 969-976

Fnigeri et Lin (1992) J. Immunol. 148: 851-867

Gu et al. (1994) Science 265: 103-106

Hooper et al. (1987) Nature 326: 292-295

Ishizaka (1989) Curr. Op. Immunol. 1: 625-629

P. Kamoun “Appareils et Méthodes en Biochimie [Apparatus and Methods in Biochemistry]”, 3^{rd} Ed. Medicine—Sciences Flammarion

Keon et al. (1990) Methods and Enzymology 185: 527-537

Kinet (1992) Curr. Op. Immunol. 4: 43-48

Koller et Smithies (1989) Proc. Natl. Acad. Sci. 86: 8932-8935

Kuster et al. (1992) J. Biol. Chem. 267: 12782-12787

Mansour et al. (1988) Nature 336: 348-352

Metzger (1992) Immunol. Rev. 125: 37-48

Miller et al. (1988) Nucl. Acids Res. 16: 215

Sambrook et al. (1989) Molecular cloning: a laboratory manual second edition—Cold Spring Harbor Laboratory Press. Cold Spring Harbor, N.Y. USA

Takizawa et al. (1992) J. Exp. Med. 176: 469-475

Truong et al. (1993) Eur. J. Immunol. 23: 3230-3235

Vangelista et al. (1999) J. Clin. Inv. 103: 1571-1578

1. A non-human transgenic animal cell, characterized in that it expresses at least one nucleotide sequence coding for at least one of the chains of the human F_{c} fragment receptors of the immunoglobulins and a nucleotide sequence coding for the heavy chain of an immunoglobulin, of which at least all or part of the F_{c} fragment is of human origin and characterized in that the animal gene coding for the chain homologous to the said chain of the human receptor is inactive.

2. The cell according to claim 1, characterized in that said nucleotide sequence coding for at least one of the human F_{c} fragment receptors is stably integrated into the genome of said cell.

3. The cell according to claim 3, characterized in that the integration of said nucleotide sequence into said genome is practiced by homologous recombination (knock-in) at the level of said animal gene homologous to the human gene coding for at least one of the human F_{c} fragment receptor, said integration provoking the inactivation of said homologous animal gene.

4. The cell according to one of claims 1 to 3, characterized in that the said nucleotide sequence coding for at least one of the human F_{c} fragment receptor chains is operationally linked to sequences for regulation of expression, said sequences controlling the expression of said nucleotide sequence in said cell.

5. The cell according to claims 1 to 4, characterized in that the said nucleotide sequence coding for the heavy chain of an immunoglobulin is the endogenous animal gene, with the exception of the sequence coding for all or part of the F_{c} fragment of said immunoglobulin that is of human origin, said sequence coding for all or part of the F_{c} fragment having been integrated into the said gene by homologous recombination (knock-in).

6. The cell according to claims 1 to 4, characterized in that the said nucleotide sequence coding for the heavy chain of an immunoglobulin is the human gene coding for the heavy chain of an immunoglobulin, said human gene being integrated by homologous recombination (knock-in) into the genome of said cell at the level of said homologous animal gene, said integration provoking the inactivation of said animal homologue.

7. The cell according to claims 1 to 4, characterized in that the said nucleotide sequence coding for the heavy chain of an IgE immunoglobulin is present in episomal form in said cell and in that the said homologous animal gene is inactive in said cell.

8. The cell according to claim 7, characterized in that said homologous animal gene is inactivated by homologous recombination (knock-out).

9. The cell according to claims 1 to 8, characterized in that said immunoglobulin is an IgE and said F_{c} fragment human receptor is a human F_{c}εR receptor on the F_{c} of IgE.

10. The cell according to claim 9, characterized in that said nucleotide sequence codes for a repertoire of heavy chains of IgE immunoglobulins.

11. The cell according to claim 9, characterized in that said nucleotide sequence coding for an IgE immunoglobulin is a mini-gene.

12. The cell according to claim 9, characterized in that said nucleotide sequence coding of the heavy chain of an IgE, of which at least all or part of the F_{c} fragment is of human origin and is operationally linked to the endogenous animal sequences for regulation transcription of the gene of the IgE heavy chain, said sequences controlling the expression of said human gene in said cell.

13. The cell according to claim 9, characterized in that said nucleotide sequence coding the heavy chain of an IgE, of which all or part of the F_{c} fragment is of human origin and is operationally linked to the endogenous human sequences for regulation of transcription of said gene of the heavy chain of the human IgE, said sequences controlling the expression of said human gene in said cell.

14. The cell according to claims 9 to 13, characterized in that the part of F_{c} fragment is composed of the C_{ε}3 and C_{ε}4.

15. The cell according to claims 9 to 14 characterized in that said F_{c}εR receptor is chosen from among the F_{c}εRI, F_{c}εRII and F_{c}εRIII.

16. The cell according to claim 15, characterized in that said F_{c}εR receptor is the F_{c}εRI receptor.

17. The cell according to claim 15, characterized in that of the polypeptide chains comprising the F_{c}εRI receptor, at least the α chain is of human origin.

18. The cell according to claims 1 to 17, characterized in that the genome of said cell contains in addition at least one reporter gene operationally linked to one or a plurality of sequences for regulation of the expression inducible following stimulation of the F_{c}εR receptor and/or stimulation of IgE synthesis.

19. The cell according to claim 18, characterized in that said reporter gene codes for an auto-fluorescence protein chosen from the group comprising green fluorescence protein (GFP), enhanced green fluorescence protein (EGFP), red fluorescence protein (RFP), blue fluorescence protein (BFP), yellow fluorescence protein (YFP) and the fluorescent variants of these proteins.

20. The cell according to claim 18, characterized in that said reporter gene coded for an enzyme detectable by a histochemical method.

21. The cell according to claim 20, characterized in that said enzyme is chosen from the group comprising β-galactosidase, β-glucuronidase, alkaline phosphatase, alcohol dehydrogenase, luciferase, chloramphenicol acetyl transferase, and growth hormone.

22. The cell according to claim 18, characterized in that said sequence(s) for regulation of expression is/are chosen from among the promoter of the interleukin-4 (IL-4) gene, the promoter of the CD23 gene, and the promoter of any other gene, whose expression is induced following stimulation of the F_{c}εR receptor and/or stimulation of IgE synthesis.

23. The cell according to claims 1 to 22, chosen from the group comprised of the cells of the mouse, the rat, the hamster, the guinea pig, the rabbit, primates, porcines, ovines, caprines, bovines, the horse.

24. The cells of the mouse according to claim 23.

25. The cell according to claims 1 to 24, characterized in that said cell is chosen from among the cells of the immune system, the neuronal cells, the embryonic stem cells, the hematopoietic stem cells, and the neuronal stem cells.

26. The cell according to claim 25, characterized in that said immune system cell is chosen from among the T lymphocytes, the NK cells, the K cells, the B lymphocytes, the mastocytes, the macrophages, the monocytes, the neutrophils, the eosinophils, the basophils, the platelets, the monocytes of dendritic cells, the Langerhans cells.

27. A stem cell according to claim 25, characterized in that said stem cell is subsequently differentiated into a cell chosen from among the immune system cells according to claim 26 and the neuronal cells.

28. A non-human, transgenic animal comprising at least one cell according to claims 1 to 27.

29. The animal according to claim 28, characterized in that it is selected from among the mouse, the rat, the hamster, the guinea pig, the rabbit, primates, porcines, ovines, caprines, bovines, the horse.

30. The animal according to claim 29, characterized in that the animal is a mouse.

31. An animal according to claims 28 to 30, characterized in that it expresses a repertoire of functional IgE immunoglobulins following exposure to at least one allergen, said IgEs having at least all or part of the F_{c} fragment of human origin and characterized in that it expresses at least one of the F_{c} fragment human receptor chains of the F_{c}εR immunoglobulins.

32. An in vitro method for demonstrating an allergen and/or determining the allergizing power of said allergen, characterized in that it comprises the steps of:
a) Placing of said allergen in contact with a cell according to one of claims 1 to 27;
b) determination if an immediate cellular and/or inflammatory reaction is produced, and
c) optionally, qualitative and/or quantitative evaluation of said immediate cellular hypersensitivity and/or inflammatory reaction.

33. An in vivo method for demonstrating an allergen and/or determining the allergizing power of said allergen, characterized in that it comprises the steps of:
a) placing of said allergen in contact with a said animal according to any one of claims 28 to 31;
b) determination if an immediate hypersensitivity and/or inflammatory reaction is produced, and
c) optionally, qualitative and/or quantitative evaluation of said immediate hypersensitivity and/or inflammatory reaction.

34. A screening method of a compound that modulates the immediate hypersensitivity and/or inflammatory reaction in a human being, characterized in that it comprises the steps of:
a) the placing in contact of a cell according to claims 1 to 27 and/or an animal according to one of claims 28 to 31 with an allergen responsible for triggering the immediate hypersensitivity and/or inflammatory reaction and simultaneously or staggered in time with said compound;
b) the placing in contact of a cell according to claims 1 to 27 and/or an animal according to one of claims 28 to 31 with said allergen of step a);
c) determination and qualitative, optionally quantitative, evaluation, if an immediate hypersensitivity and/or inflammatory reaction is produced and then comparison of said immediate hypersensitivity and/or inflammatory reactions triggered in a) and b);
d) then, identification of the compound that selectively modulates the immediate hypersensitivity and/or inflammatory reaction.

35. A method according to any one of claims 32 to 34, characterized in that said determination and/or evaluation of said immediate and/or inflammatory reaction is practiced by measuring the IgE level synthesized by said cell according to one of claims 1 to 27 and/or by the level of serum IgE of the animal according to one of claims 28 to 31.

36. The method according to any one of claims 32 to 34, characterized in that said determination and/or evaluation of the immediate hypersensitivity and/or inflammatory reaction is practiced by the detection and/or measurement of the rate of expression of a so-called reporter gene.

37. The method according to claims 32 to 36, characterized in that said immediate hypersensitivity and/or inflammatory reaction is chosen from among systemic anaphylaxis, cutaneous anaphylaxis, asthma, eczema, rhinitis, urticaria, hay fever, atopic dermatitis, the chronic inflammatory intestinal diseases (CIID) and/or colo-rectal diseases, the parasitic diseases in which an IgE response is known to be protective, especially the helminthic parasitic diseases (infections by Schistosoma mansoni and Nippostratus filariae), food allergy, household dust allergy.

38. Use of a cell according to claims 1 to 27 and/or an animal according to claims 28 to 31 for analysis and study of the molecular, biological, biochemical, physiological and/or pathophysiological mechanisms of the immediate hypersensitivity and/or inflammatory reaction.
